(19) United States
(12) Patent Application Publication
(10) Pub. No.: US 2005/0055070 A1
(43) Pub. Date: Mar. 10, 2005

(54) Method and device for treatment of skin conditions

(76) Inventors: Gareth Jones, Pontyclun (GB); Kenneth Board, Swansea (GB); Gareth Peter Evans, Swansea (GB)

Correspondence Address: Robert C. Klinger;Jackson Walker LLP, Suite 600, 2435 N. Central Expressway, Richardson, TX, 75080, US

(21) Appl. No.: 10/380,189

(22) Filed: Mar. 7, 2003

(51) Int. Cl. A61N 5/06

(52) U.S. Cl. 607/88; 607/89

(57) ABSTRACT

Method and device for treatment of skin conditions A method and apparatus for improving the cosmetic appearance of a region of skin 11 affected by Acne Vulgaris, Rosacea or similar skin condition by means of directing light radiation 12 from an illuminating device 1 on to the skin 11. The apparatus 10 comprises a control unit 9 that operates one or more LEDs 7 (light emitting diodes) of the illuminating device 1. Each dose of light radiation 12 lasts for at least 100 ms, during which time the skin 11 receives light energy from the LED(s) 7, which causes a photochemical reaction that stimulates the production of free radicals (singlet oxygen) that react with, and at least partially disable or destroy, bacteria that contribute to the symptoms of the skin condition. The light energy directed on to the skin 11 during any given period of 10 μs is less than 0.5 Jcm⁻², and during any given period of 100 ms is less than 5 Jcm⁻². Substantially no beneficial photo-thermal reaction occurs within the skin 11. Light having wavelengths around 405 nm and/or 585 nm is used. The duration of a single dose may be much longer than 100 ms and can last up to 10 hours (for overnight treatment).

### BACKGROUND OF THE INVENTION

1. Field of the Invention

The present invention relates to a method and apparatus for the treatment of a skin condition or for improving the cosmetic appearance of skin. In particular, the invention relates to a method and apparatus for the treatment of Acne Vulgaris.

Acne Vulgaris is a skin condition which affects almost 100% of the population at some point in their lives. It is a condition of the sebaceous follicles (pores) and can lead to lesions on the skin, primarily on the face, shoulders and back. The symptoms of Acne Vulgaris, and of other similar skin conditions, can be unsightly and undesirable cosmetically.

Acne Vulgaris is caused by a number of factors, of which the most significant are believed to be: excessive hormone production (especially androgens), excessive sebum production (sebum is an oily substance produced by the sebaceous glands to keep the skin soft, pliable and waterproof), excessive dead cell shedding, the presence of bacteria (particularly Propionibacterium acnes) in the sebaceous follicles and the bodies inflammatory response (chemotaxis).

The process starts when dead skin cells and the oily substance sebum combine to block the skin's sebaceous follicles. The dead skin and sebum form a plug which traps oil and bacteria within the sebaceous follicles. The sebaceous follicle begins to swell as the as the skin continues its normal oil production. Normal skin bacteria Propionibacterium acnes multiply rapidly in the clogged pore. The multiplication of the bacteria produces substances which cause inflammation in the follicle and surrounding skin. The body's response is to send white blood cells to the inflamed areas.

The lesions range in severity and can be defined as comedones, papules, nodules, pustules and cysts. Comedones are sebaceous follicles that have become blocked. Papules are small (less than 5 mm) solid lesions slightly above the surface of the skin. Nodules are larger papules (greater than 5 mm). Pustules are dome shaped fragile lesions typically containing a mixture of white blood cells, dead cells and bacteria. Cysts are similar to pustules but are larger and are severely inflamed and often lead to scarring.

Various drug treatments are known to be at least partially effective in preventing Acne Vulgaris.

1. Description of the Related Art

A treatment for Acne Vulgaris using light radiation is disclosed in GB 2 368 020. In the embodiments disclosed, radiation is provided at specific energy densities and wavelengths in order to cause photo-chemical and photo-thermal reactions in the skin. The photo-chemical reaction leads to a partial disabling or eradication of a cause of the skin condition while the photo-thermal reaction increases collagen production, thereby helping to reduce the risk of scarring.

The method disclosed in GB 2 368 020 requires the provision of relatively high doses of radiation (0.5 Jcm⁻² to 5 Jcm⁻²) in short periods of time (10 μs to 100 ms) in order to produce the photo-thermal effects in the skin. Providing these high energy levels over short periods of time requires high power lasers. These are expensive to manufacture and operators may be required to have specialist training and knowledge to use them safely and effectively.

### SUMMARY OF THE INVENTION

It is an object of the invention to provide an apparatus for the treatment of a skin condition that is relatively inexpensive and/or simple to operate and/or to provide a method for the treatment of a skin condition that is relatively inexpensive and/or simple to carry out.

According to an aspect of the invention there is provided a method for improving the appearance of a region of skin by means of conducting the steps of providing an illuminating device, and operating the illuminating device to direct light radiation on to the region of skin, wherein during a period of at least 100 ms, said region receives light energy from the illuminating device of at least 0.01 Jcm⁻², the light energy so delivered in any given period of 10 μs is less than 0.5 Jcm⁻², and the light energy so delivered in any given period of 100 ms is less than 5 Jcm⁻².

According to another aspect of the invention there is provided a method for treating the skin condition Acne Vulgaris, the method including the steps of providing one or more light emitting diodes, and operating said one or more light emitting diodes to direct light radiation on to a region of skin affected by Acne Vulgaris, wherein during a single dose lasting at least 100 ms, said region receives light energy from said one or more light emitting diodes of at least 0.01 Jcm⁻², and the light energy causes a photochemical reaction in said region that stimulates the production of free radicals that react with, and at least partially disable or destroy, bacteria that contribute to the symptoms of Acne Vulgaris.

According to yet another aspect of the invention, there is provided a method of improving the cosmetic appearance of a region of the skin of a mammal comprising the steps of providing an illuminating device, and operating the illuminating device to direct light radiation on to the region of skin, wherein during a period of at least 100 ms, said region receives light energy from the illuminating device of at least 0.01 Jcm⁻², the light energy so delivered in any given period of 10 μs is less than 0.5 Jcm⁻², and the light energy so delivered in any given period of 100 ms is less than 5 Jcm⁻².

The light energy so delivered advantageously causes a photochemical reaction within the skin. Thus the cosmetic appearance of the affected area of skin may be improved.

By providing direct light radiation for a longer period of time than prior art methods, similar photon or energy densities can be delivered but at much lower power densities or energy flux densities, thereby reducing the likelihood of any adverse reactions of the skin to the radiation, for example, causing photo-mechanical effects (explosive expansion of the tissue) or photo-thermal effects (rapid heating of the tissue). In addition, providing radiation at lower power means the radiation is less likely to induce erythema (redness of the skin). Advantageously, the method of the invention is performed without any active pre-cooling of the skin immediately before treatment. Some prior art methods utilising higher light intensities than in the present invention cause the target area to be heated so quickly that heat cannot be effectively dissipated by means of the vascular system, such methods thus requiring cooling of the skin before treatment to prevent undesirable effects associated with the over-heating of the skin.

Preferably, during said period of at least 100 ms, said region receives light energy from the illuminating device of less than 10 kJcm⁻², and more preferably less than 1 kJcm⁻². Preferably, during said period of at least 100 ms, no more than 100 Jcm⁻² of light energy is delivered over any given period of 1 minute. The method may be so performed that during said period of at least 100 ms, no more than 100 Jcm⁻² of light energy is delivered over any given period of 10 minutes. The light energy so delivered in any given period of 1 ms is preferably less than 1 Jcm⁻². The light energy so delivered in any given period of 10 ms is preferably less than 1 Jcm⁻². The light energy so delivered in any given period of 500 μs is preferably less than 1 Jcm⁻². Said period is preferably less than 10 hours.

The light energy so delivered in any given period of 10 μs is preferably less than 50 mJcm⁻², more preferably is less than 10 mJcm⁻², and yet more preferably is less than 5 mJcm⁻². The light energy so delivered in any given period of 10 μs may be less than 1 mJcm⁻².

The lower power requirements of the present invention compared to prior art methods means that LEDs (Light Emitting Diodes) can be used rather than lasers. At low powers, these tend to be cheaper and less complicated than equivalent laser systems. Also the use of laser devices is in many countries subject to strict regulations. The illuminating device thus advantageously includes a plurality of LEDs, for example, including a plurality of LEDs grouped together to form an array. A single LED may be used. For example, the LEDs may form a 1 dimensional line array or a 2 dimensional array suitable for illumination of larger areas such as the face or back. Advantageously, the LEDs could be grouped to form a face mask under which a user (i.e. a person whose skin is affected by a skin condition) could be positioned. Using such a face mask system enables a substantially uniform dose of radiation to be applied to the whole face within a short amount of time, for example in a period as short as 30 seconds. In contrast, it can take up to 30 minutes for an operator to treat a whole face using the single spot applicator system. In both cases, the skin would receive a similar dose of light radiation to induce a similar photochemical response.

The method may be utilised in a method of therapeutic treatment on the human body. The method may be in the form of a method of cosmetic treatment.

The method is preferably for the cosmetic treatment of Acne Vulgaris. The method may be in the form of a method for the cosmetic treatment of Rosacea.

The “skin” referred to may be the skin of a mammalian animal, preferably human. The method is preferably non-surgical. For example, the illuminating device is preferably arranged and configured so as to be unable to be operated at sufficiently high power to be considered a surgical device necessitating a skilled operator, such as a surgeon. The illuminating device may thus be arranged to be intrinsically more safe and less complex to operate and manufacture than the pulsed dye laser of apparatuses of the prior art.

Advantageously, a photo-chemical reaction is caused that disables or destroys, wholly or partially, the bacteria Propionibacterium acnes, which, as described above, is one of the causes of Acne Vulgaris. Propionibacterium acnes is anaerobic and is harmed by the presence of oxygen. The photo-chemical reaction may be such that the symptoms of acne are, at least temporarily, reduced without necessarily permanently destroying the agents, for example the bacteria, that contribute to the symptoms of a skin condition.

The photo-chemical reaction may take place in a substance as a result of that substance absorbing radiation within a range of particular wavelengths (the reaction being significantly slower or non-existent outside the range). Preferably the wavelength of radiation used produces a photo-chemical reaction in a substance (a chromophore, for example) of, on or in the skin that results in the production of free radicals (for example in the form of oxygen singlets) which thereafter may destroy the bacterium. The chromophore targeted is preferably porphyrin. Porphyrin is a naturally occurring substance produced by the bacteria Propionibacterium acnes. Porphyrin produces singlet oxygen when excited by light of a wavelength of around 585 nm (yellow light) and also when excited by light of a wavelength of around 405 nm (violet/near ultra-violet light). Light at other wavelengths is also able to stimulate free-radical production.

Thus by exciting Porphyrin in the manner outlined above it is possible to disable or destroy the bacterium responsible for Acne Vulgaris in a pain-free, non-invasive and efficient manner. The method according to the present invention provides a means of destroying, at least partially, the bacteria that contributes to a skin condition but without needing to use high power lasers, which as mentioned above have various disadvantages.

The duration of the treatment or the length of the period during which light radiation is provided by the illuminating device may, for example, be between 100 ms and 30 minutes, is preferably between 200 ms and 10 seconds, is more preferably between 200 ms and 3 seconds and is yet more preferably between 300 ms and 2 seconds. The duration or period may alternatively or additionally be greater than 500 ms. The delivery of light radiation may continuous during a single treatment or during said period. Alternatively, the delivery of light radiation may be pulsed during a single treatment or during said period.

The duration of the light radiation is preferably substantially greater than the thermal relaxation time of the microvascular system near the area of skin affected by the skin condition. Thus, the photo-thermal response taught in GB2368020 is not significant in the proposed method due to the relatively low dose of light radiation energy supplied within the hundreds of micro-seconds timescale (of the same order of time as that of the microvascular thermal relaxation timescale).

The energy density of the illuminating radiation delivered may, for example, be between 0.01 and 100 JCm⁻², is preferably between 0.1 and 10 JCm⁻², is more preferably between 0.5 and 3 JCm⁻² and is even more preferably between 1 and 3 Jcm⁻². The energy density may be less than 2 JCm⁻². Lower energy densities are preferable at longer durations.

The dominant wavelength of the illuminating radiation is preferably pre-determined. The radiation may, for example, include radiation having a wavelength of between 350 nm and 1500 nm, or more preferably between 350 nm and 1000 nm. The wavelength is preferably between 350 nm and 700 nm, more preferably between 570 nm and 600 nm and is even more preferably between 580 nm and 590 nm. The illuminating radiation may include radiation substantially concentrated around the wavelength of yellow light (585 nm). The radiation may include radiation having a wavelength of between 350 nm and 450 nm, or more preferably between 390 nm and 420 nm. The illuminating radiation may include radiation substantially concentrated around the wavelength of violet/near ultra-violet light (405 nm). In accordance with the invention, especially insofar as the treatment for Acne Vulgaris is concerned, the radiation may be chosen to correspond to a photosensitizer such as for example porphyrin in skin tissue. The wavelength of the light radiation may be chosen to correspond with a wavelength suitable for targeting the porphyrin in the skin layers at a depth suitable to ensure that singlet oxygen is released which affects the proprionibacterium Acnes without significantly affecting other tissues.

It is especially advantageous to use radiation at one or more wavelengths that correspond to one or more of the peaks of the porphyrin absorption curve.

The illuminating radiation may be provided to an area of the affected skin of between 12 and 200 mm², for example, to a spot size of diameter 4-16 mm. The area may be less than 100 mm². A greater area may be treated however. For example an area of up to 0.1 m² or even up to 0.5 m² might be treated simultaneously.

Preferably the illuminating radiation delivered has a peak optical power level of less than 100 Wcm⁻² and more preferably less than 10 W cm⁻². The illuminating radiation preferably provides a peak optical output power level of between 1 and 5 Wcm⁻¹.

The light energy so delivered by the illuminating device may cause a photochemical reaction within the affected skin thereby stimulating the production of free-radicals, which react with, and at least partially disable or destroy, agents causing the skin condition.

A low power spot or line treatment may be used to “top up” the higher dose treatment described above. The top up treatment may for example be provided at lower powers than the higher dose treatment and over longer periods of time (for example, overnight). Such a low power treatment might be particularly well suited to use of the illuminating device in the home.

The method is preferably performed such that the distance between the illuminating device and the surface onto which the radiation is delivered is less than 1000 mm, and is preferably less than 100 mm. The distance of separation may be less than 50 mm. The illuminating device and the surface may be directly adjacent to each other and may for example touch when radiation is being delivered.

According to another aspect of the invention there is also provided an apparatus for the treatment of a skin condition comprising an illuminating device, and a control unit for controlling the operation of the illuminating device, wherein the illuminating device is so arranged and configured that it is able in use to emit light radiation of an energy and wavelength profile sufficient to cause a photochemical reaction within an area of skin affected by a skin condition, which reaction would result in agents causing the skin condition being at least partially disabled or destroyed, and the control unit and illuminating device are so arranged and configured that the control unit is able in use to cause the illuminating device to direct light radiation on to an area within a distance of no more than 1000 mm from the illuminating device such that: the light energy received at said area during a period of at least 100 ms is at least 0.01 Jcm⁻², the light energy received at said area in any given period of 10 μs is less than 0.5 Jcm⁻², and the light energy so delivered in any given period of 100 ms is less than 5 Jcm⁻², whereby the apparatus may be used to treat the skin condition.

According to a further aspect of the invention there is also provided an apparatus for the treatment of Acne Vulgaris comprising one or more light emitting diodes, and a microprocessor for controlling the operation of said one or more light emitting diodes, wherein said microprocessor and said one or more light emitting diodes are so arranged and configured that said one or more light emitting diodes are able in use to deliver a dose of light radiation onto a surface during a period of at least 100 ms, such that the surface receives during said dose light energy of at least 0.01 Jcm⁻², the light so delivered including radiation having a wavelength in the range of 350 nm and 700 nm, whereby the apparatus may be used to treat a region of skin affected by the condition Acne Vulgaris by causing a photochemical reaction in said region that stimulates the production of free radicals that react with, and at least partially disable or destroy, bacteria that contribute to the symptoms of Acne Vulgaris.

Further optional or preferred features of the apparatus according to either or both of the above-mentioned aspects of the invention are described below.

The apparatus may alternatively or additionally be suitable for improving the cosmetic appearance of the skin of a mammal, for example a human being.

The illuminating device of the apparatus of the invention may be so arranged and configured that it is able, when being used to direct light onto the skin, to emit light radiation of an energy and wavelength profile sufficient to cause a photochemical reaction within an area of the skin being targeted. The photochemical reaction may be caused in such a way that it partially disables or destroys agents in the skin that are causing the cosmetic appearance of the skin to be worsened.

The illuminating device preferably comprises one or more light emitting semiconductor devices. The or each semiconductor device may be in the form of a diode. The illuminating device may for example comprise one or more LEDs. LEDs are, advantageously relatively inexpensive and simple to operate in comparison to lasers. Laser diodes may additionally or alternatively be used. Conveniently, the illuminating device is in the form of a device, for example comprising at least one semiconductor device that in use acts as the active light emitting element(s), that has a power input requirement of less than 500 W, and preferably less than 100 W, per individual semiconductor device.

The control unit and illuminating device of the apparatus may be so configured and arranged that the control unit is able in use to cause the illuminating device to deliver light energy of between 0.01 and 100 Jcm⁻² to said area during a period of between 200 ms and 3 seconds, or more preferably during a period of between 300 ms and 2 seconds. Of course, the control unit and illuminating device may be arranged to emit radiation during a single treatment over a longer period of time, so that during a single treatment more than 100 Jcm⁻² is delivered over a period of greater than 3 seconds. A single treatment might last as long as up to 10 hours. Such a treatment might for example be provided overnight. Preferably, the control unit and illuminating device of the apparatus are so configured and arranged that less than 100 Jcm⁻² of light energy is delivered during any period of 3 seconds.

The control unit and illuminating device may be so configured and arranged that the illuminating device delivers pulsed light radiation during a single treatment. Alternatively, the control unit and illuminating device may be so configured and arranged that the illuminating device delivers continuous light radiation during a single treatment. The apparatus may be so configured to be able to deliver either continuous or pulsed radiation at the choice of the user.

The control unit and illuminating device of the apparatus may be so configured and arranged that the control unit is able in use to cause the illuminating device to deliver light energy of between 0.5 Jcm⁻² and 3 Jcm⁻² to said area during a period of between 100 ms and 100 seconds. The control unit and illuminating device may be so configured and arranged that the control unit is able in use to cause the illuminating device to deliver a single dose of light radiation to an area of skin, the single dose being provided over a period of between 200 ms and 10 seconds (or more preferably between 200 ms and 3 seconds) and the energy of the light radiation delivered during the single dose being greater than 0.1 Jcm⁻² and being equal to T₁×P₁, where T₁=the length in time of the single dose and P₁ has the units of optical power density (power per unit area) and satisfies 0.2 Wcm⁻²<P₁<20 Wcm⁻².

The apparatus may be in the form of a top up apparatus allowing lower levels of light energy to be delivered. For example, the control unit and illuminating device may be so configured and arranged that the control unit is able in use to cause the illuminating device to deliver a single dose of light radiation to an area of skin, the single dose being provided over a period of between 300 ms and 10 seconds (or more preferably between 300 ms and 3 seconds) and the energy of the light radiation delivered during the single dose being equal to T₂×P₂, where T₂=the length in time of the single dose and P₂ has the units of optical power density (power per unit area) and satisfies 0.01 Wcm⁻²<P₂<1 Wcm⁻². P₂ may satisfy the condition 0.1 Wcm⁻²<P₂<0.5 Wcm⁻².

The apparatus may be so configured and arranged that it is suitable for treatment of relatively small areas at a time. For example, the apparatus may be so configured and arranged that, during a single dose of light radiation, an area of skin of between 12 and 200 mm² is treated.

The apparatus may be so configured and arranged that it is suitable for lower power operation over longer periods of time. For example, the control unit and illuminating device may be so configured and arranged that the control unit is able in use to deliver, during a single treatment lasting between 300 ms and 10 hours (more preferably between 10 seconds and 8 hours), light radiation to an area of skin, the energy of the light radiation delivered during the single treatment being greater than 0.1 Jcm⁻² and being equal to T₃×P₃, where T₃=the length in time of the single treatment and P₃ has the units of optical power density (power per unit area) and satisfies 0.5 mWcm⁻²<P₃<500 mWcm⁻². The longer treatment may last for at least 30 seconds, preferably lasts for at least 10 minutes and more preferably lasts for at least an hour.

The apparatus may be so configured and arranged that it is suitable for treatment of relatively large areas at a time. For example, the apparatus may be so configured and arranged that, during a single treatment, an area of skin of between 0.003 m² and 0.5 m² is treated. Such an apparatus is preferably arranged such that the energy of the light radiation delivered during a single treatment is equal to T₃×P₃, where T₃=the length in time of the single treatment and P₃ has the units of optical power density (power per unit area) and satisfies 0.5 mWcm⁻²<P₃<500 mWcm⁻².

The illuminating device is preferably arranged to provide light radiation including radiation having a wavelength of between 350 nm and 1500 nm. The illuminating device is preferably arranged to emit radiation at a wavelength between 350 nm and 1000, more preferably between 350 nm and 700 nm. The illuminating device is preferably arranged to provide light radiation including radiation having a wavelength between 570 nm and 600 nm. The illuminating device may alternatively or additionally be arranged to provide light radiation of a wavelength between 390 nm and 420 nm, and preferably includes light radiation having a wavelength of about 405 nm. The Porphyrin activation spectrum has peaks at both about 585 nm and 405 nm. Providing light radiation including light having a wavelength between 390 nm and 420 nm and including light having a wavelength between 570 nm and 600 nm is considered to be particularly advantageous as such a combination may activate Porphyrin at different depths in the skin tissue.

Also, it is believed that red light (of a wavelength between 630 nm and 680 nm) may aid in the wound healing process.

The illuminating device is preferably arranged to provide light radiation having a peak power level of less than 100 Wcm⁻². More preferably, the illuminating device is preferably arranged to provide light radiation having a peak power level of less than 10 Wcm⁻². Even more preferably, the illuminating device is preferably arranged to provide light radiation having a peak power level of less than 5 Wcm⁻². Advantageously, the peak power level is between about 1 Wcm² and 5 Wcm⁻². The peak power level may be between about 1 Wcm⁻² and 3 Wcm⁻².

The apparatus of the invention is advantageously so arranged that the apparatus may be used to treat the skin of a patient without the need to pre-cool the skin before treatment.

The apparatus may include cooling means for controlling the temperature of the illuminating device. The control unit is preferably arranged to control operation of any such cooling means. The control unit may conveniently comprise a suitably pre-programmed microprocessor. The cooling means may include a Peltier device. The cooling means may additionally or alternatively operate by utilising liquid coolant.

Above, mention is made of light energy causing a photochemical reaction within the skin. In order to cause a photochemical reaction in the skin, it is believed that the extent/amount of the photochemical reaction depends primarily on the amount of light (i.e. number of photons) received per unit area and that the power of light used has a lesser effect. According to certain embodiments of the invention, it is preferred for the light energy to be delivered over a relatively long period of time. According to certain other embodiments of the invention, it is preferred for the light energy to be delivered over a relatively short time without exceeding a given power level that might cause undesirable effects resulting from heating of the skin. Thus according to some aspects of the invention it is preferred to have as high a power output as is reasonably possible without exceeding a maximum power output, above which there would be a risk of causing such undesirable effects. It is considered especially advantageous that the present invention is able to provide an apparatus that utilises semiconductor light emitting devices (such as LEDs or laser diodes) that are able to operate at such power levels.

In accordance with the present invention there is also provided a use of said apparatus including illuminating a surface within a distance of no more than 1000 mm from the illuminating device with radiation of an energy such that an area of at least 12 mm² receives energy from the illuminating device during a period of at least 100 ms of at least 0.01 Jcm⁻², the radiation including radiation having a wavelength of between 350 and 700 nm (preferably including radiation having a wavelength of between 570 nm and 600 nm and/or of between 390 nm and 420 nm), the energy received from the illuminating device at said area in any given period of 10 μs being less than 0.5 Jcm⁻², and the energy received from the illuminating device in any given period of 100 ms being less than 5 Jcm⁻². Said use is advantageously, but not necessarily, performed to treat a skin condition on the skin of a human. The use of the apparatus may for example be in the form of performing the method of the invention. The use of the apparatus may for example be in the form of testing and/or demonstrating the apparatus on a surface that is, for example, not in the form of the skin of a living animal or human.

It will be appreciated that the method of the invention may include use of the apparatus of the invention and that the apparatus of the invention may be arranged and configured to be suitable for performing the method of the invention. Thus, features described with reference to the method of the invention may be incorporated in the apparatus of the invention. Also, features described with reference to the apparatus of the invention may be incorporated in the method of the invention.

### BRIEF DESCRIPTION OF THE DRAWINGS

Embodiments of the present invention will now be described by way of example with reference to the following schematic drawings of which:

FIG. 1 shows an apparatus according to a first embodiment including a control unit and an illuminating device being used to treat the skin of a patient;

FIG. 2 shows the control unit and illuminating device of the apparatus shown in FIG. 1;

FIG. 3 shows in greater detail the illuminating device of the apparatus shown in FIG. 1;

FIG. 4 shows a block diagram illustrating the components of an apparatus according to a second embodiment of the invention;

FIG. 5a is a sectional side view of a hand-piece of the apparatus according to the second embodiment;

FIG. 5b is a plan view of the hand-piece shown in FIG. 5a;

FIG. 6a is an end-on view of the hand-piece of FIG. 5a showing an LED assembly;

FIG. 6b is a perspective view of the LED assembly shown in FIG. 6a; and

FIG. 6c is a side view of the LED assembly shown in FIG. 6a.

### DETAILED DESCRIPTION OF THE PREFERRED EMBODIMENTS

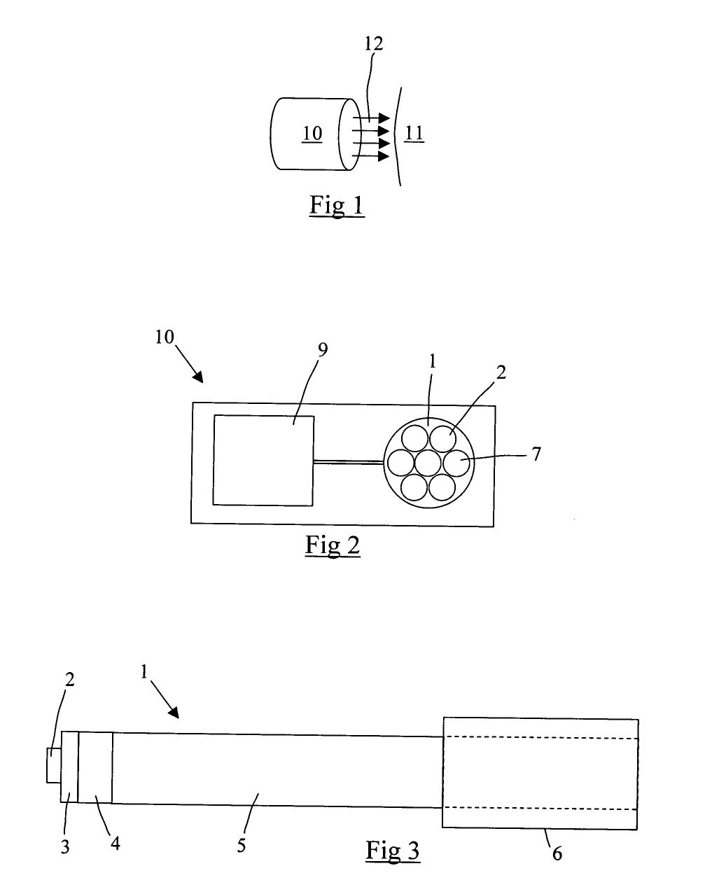

FIG. 1 shows an apparatus 10 according to a first embodiment of the present invention for the treatment of a skin condition such as acne by directing light radiation 12 onto the skin 11 of a human patient. The skin to be treated in this embodiment is an area of skin on the face including a spot having a diameter about 6 mm. The apparatus 10, in this embodiment a hand-held battery powered unit, includes an illuminating device 1 and a control unit 9 linked thereto which controls the radiation emitted by the device 1. The housing of the apparatus 10 is elongate is shape and has a proximal end via which light is emitted from the illuminating device 1. The overall length of the housing is about 15 cm.

The apparatus 10, in use, is placed against the skin with the illuminating device 1 being positioned so as to direct radiation towards the affected area. Before operation the apparatus is programmed to set the duration of the radiation and the power of radiation. In this embodiment the apparatus is set to provide a single pulse of light energy lasting 1 second that delivers 1.5 Jcm⁻² to the 6 mm diameter spot. The peak power output of the illuminating device 1 is below 5 W/cm⁻². The energy profile over time of the radiation delivered is such that the energy is continuously delivered during the 1 second pulse and is such that during any period of 10 μs the light energy delivered is less than 0.5 Jcm⁻² and such that during any period of 100 ms the light energy delivered in is less than 5 Jcm⁻². The method of this embodiment relies solely on photochemical effects that occur within the skin as is explained in further detail below. In general, it is preferable that the method of the invention is so performed that, and/or the apparatus of the invention is so arranged that in normal use, there is substantially no beneficial photo-thermal reaction caused within the skin.

The radiation received by the skin 11 causes a photochemical reaction in Porphyrin in the skin that releases singlet oxygen (a free radical), which then destroys at least some of the bacteria, which is one of the causes of the symptoms of acne. The radiation received is however well below the level at which erythema may be induced. The radiation emitted by the illuminating device includes light having an intensity that peaks at a wavelength of about 585 nm and includes components of light radiation having wavelengths in the range of 570-600 nm. Such wavelengths are suitable for targeting the porphyrin in the skin layers at a depth sufficient for causing the released reactive oxygen to affect the Propionibacterium acnes bacteria without significantly affecting other tissues.

The illuminating device includes a plurality of LEDs 7 arranged in a 2-D array 2 (shown schematically in FIG. 2 as LEDs arranged in a close-packed formation) connected to a lens arrangement (not shown) that focuses the radiation emitted by the LEDs, so that a concentrated source of light is provided. The device 1 is therefore suitable for “spot treatment” of skin condition (i.e. treating small areas one at a time). FIG. 3 shows other components of the illuminating device 1, such other components being provided to cool the LEDs.

Referring to the FIG. 3, there is shown illuminating device (generally designated 1) comprising, in sequence, an LED diode array 2, a high thermal conductivity heat spreader layer 3, a Peltier type thermoelectric cooler 4 and a heat pipe arrangement 5 (including a distal condenser 6).

The heat spreader 3, thermoelectric cooler 4 and heat pipe arrangement 5 are provided to keep the operating temperature of the LEDs at a reduced level and therefore operating most efficiently. It is well-known that the efficiency of an LED increases with reduced operating temperature and in the case of LEDs operating at wavelengths between 550 nm and 650 nm this dependence on temperature is very high.

Heat flowing from the LED diode array 2 is spread over a larger area by the high conductivity spreader layer 3. This layer is typically only a few millimetres thick and provides rapid and highly efficient heat transfer away from the diode array 2. Heat then flows into the cold end of the thermoelectric Peltier cooler 4. The hot end of the thermoelectric Peltier cooler layer 4 is in heat transfer coupling with the heat pipe 5. The high thermal conductivity layer 3 includes a diamond material, which is laid down by means of a plasma/chemical vapour deposition method.

The Peltier cooler 4 includes a separate control means including associated drive circuitry which accurately controls, during use, the heat transfer away from the LED diode array 2 via the high thermal conductivity spreader layer 3. Accurate control of the driven Peltier thermoelectric cooler 4 (in combination with the provision of the high thermal conductivity heat spreader layer 3 and the downstream heat pipe cooling arrangement 5) provides for extremely efficient thermal management of the apparatus, and in particular the diode array 2, which ensures consistency of the light output.

The heat pipe arrangement 5 includes a wick to direct fluid coolant (contained in the heat pipe arrangement 5) away from the “hot zone” via capillary action, gravity or diffusion. The arrangement includes a fluid return system to return cooled fluid from the “cold zone” at the distal end of the apparatus, which is provided with a condenser 6. The condenser 6 is itself cooled by air cooling.

The treatment of Acne using this method has been/will be trialled on patients suffering from facial acne. The illuminating device used in these trials was in the form of a small spot illuminating device (as described above). During the trials, the radiation emitted during a single dose was about 1.5 J/cm⁻² for a 6 mm spot size. Trials are also planned with the use of the illuminating device similar to that described above but being in the form of a larger 2-D array of such devices. Such an array of devices would for example be suitable for illumination and treatment of larger areas such as the face or back. The results of the initial trials appear to demonstrate a beneficial effect on the skin conditions treated.

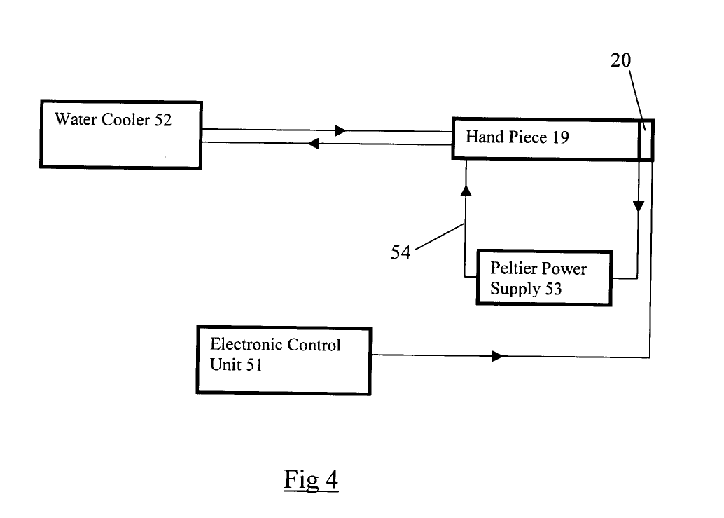

An alternative, and preferred, apparatus 18 for performing the above described method is illustrated by FIGS. 4 to 6c, which show an apparatus according to a second embodiment of the invention.

FIG. 4 shows a block diagram illustrating schematically the parts of the apparatus 18. The apparatus 18 includes a hand-piece 19 in which is housed an LED assembly 20 with an associated integral cooling system (not shown in FIG. 4), a control unit 51 for controlling the hand-piece 19, a power supply 53 for the integral cooling system and a separate water cooling system 52 that removes the heat from the integral cooling system.

The electronic control unit 51 provides the electrical power supply to each LED of the LED assembly in a controlled manner in the form of continuous DC (direct current) power or pulsed power.

The water cooling system 52 comprises a submersible pump, a water reservoir and a radiator system. The radiator system receives heated water from the integral cooling system of the hand-piece 19. That water cools as it passes through the radiator. The cooler water is them fed back to the integral cooling system of the hand piece 19. Heat exchange at the radiator is assisted by means of an air fan.

The power supply 53 for the integral cooling system unit incorporates a feedback loop 54 that assists in the cooling method employed. The temperature of the LED assembly 20 is sensed and the power delivered to the cooling system is controlled to be dependent on the temperature so sensed in order to keep the temperature of the LED assembly at a pre-selected temperature. In this embodiment the pre-selected temperature is −15 degrees Centigrade (258K).

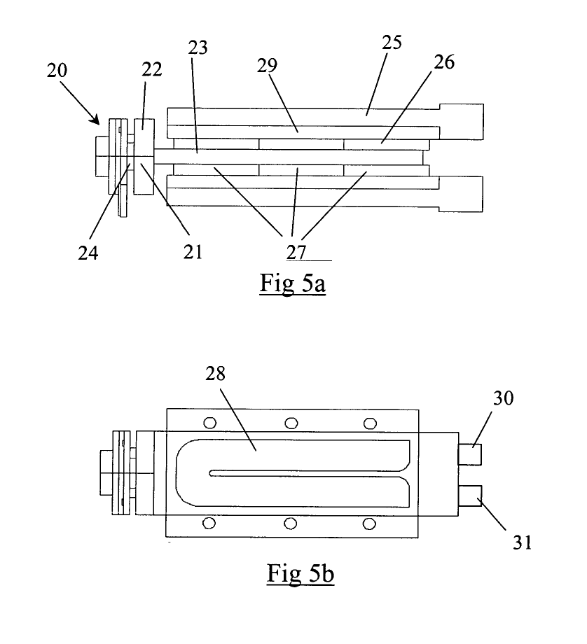

FIG. 5a shows a sectional side view of the hand piece 19 and FIG. 5b shows a plan view of the hand piece 19. As mentioned above, the hand piece comprises an LED assembly 20, which is mounted at one end of the generally elongate hand piece 19, and an integral cooling system, which is housed in the main body of the hand piece. The cooling system comprises a heat spreader 21, a Peltier assembly 26 and a water-cooling unit 25. The overall length of the hand piece is about 15 cm.

The heat spreader 21 consists of a disc 22, one side of which is in thermal conductive contact with a heat sink of the LED assembly 20 and the other side of which is integrally formed with and connected to one end of a flat plate 23. The heat spreader is made from copper (but could be made from or coated with any other material having a high thermal conductivity such as silver or diamond).

The Peltier assembly 26 comprises six Peltier cooling units 27 mounted three on each side of the flat plate 23, so that the cool side of each Peltier unit 27 is in contact with the plate 23 of the heat spreader 21.

The water cooling unit 25, which partially surrounds the Peltier assembly, is in close thermal conductive contact with the hot side of each of the Peltier units 27 and, in use, removes heat from the Peltier assembly 26. The cooling unit 25 comprises two aluminium blocks, positioned on opposite sides of the hand piece 19. FIG. 5b shows one of the blocks in plan view. The block includes a duct 28 sealed by a sealing plates 29 disposed between the duct and the Peltier units 27. Relatively cool water from the separate water cooling system 52 passes into each duct 28 via an inlet port 30 and relatively warmer water is passed out of the duct 28 via an outlet port 31 and flows back to the separate water cooling system 52. The water is circulated by means of the pump of the separate water cooling system 52.

Thus, during use, the LED assembly is cooled by means of the integral cooling system and in particular by the Peltier assembly, and the Peltier assembly is cooled by means of the water cooling unit 25 and the separate water cooling system 52.

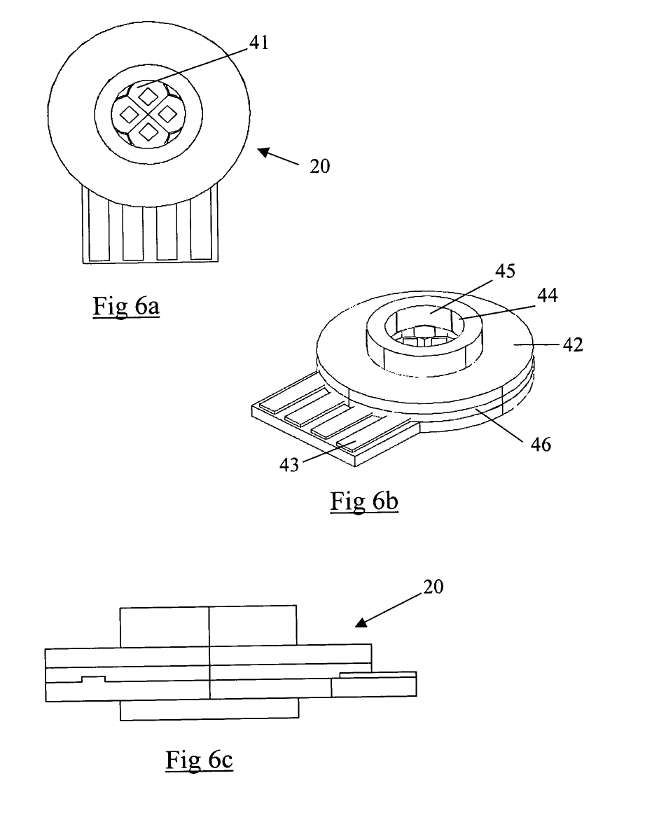

The LED assembly is shown in more details in FIGS. 6a to 6c. The LED assembly comprises four standard LEDs, each of which having been modified by shaving or machining away a part of the housing of the LED to form two adjacent perpendicular faces. A shaved face of one LED abuts a shaved face of an adjacent LED, the four LEDs thereby forming an array 41 in the general shape of a cloverleaf. By removing material from the LED housing in this way, the respective dice of the LEDs are brought into closer proximity than would otherwise be possible.

Electrical connections are provided through a printed circuit board 43, which is mounted on the flange defined by the LED assembly 20. The light output side of the LED array 41 is surrounded by a cylindrical tube, the interior of which forms a polished reflecting surface 42 which acts to direct the light from the LED array 41 through the circular aperture formed by the open end of the tube 44.

The reflecting surface 42 of the tube is so shaped as to transmit light from the LED array 41 to the circular aperture in as efficient a manner as possible. The wall of the tube 44 is arranged at such an angle that an optimum amount of light is coupled out of the LED array to the circular aperture, whilst minimizing the aperture diameter so as to achieve high optical power densities. The interior of the tube is filled with a soft transparent gel, which prevents condensation on the LED dice. The type of gel used preferably does not discolour with age or temperature cycling is preferably flexible and able to conduct some heat away from the LEDs. The gel, having a refractive index of about 1.5, provides a refractive index step between the semiconductor LED surface layers (refractive index about 3) and the air (refractive index of 1.00). This refractive index step improves the optical extraction by increasing the photon escape probability from within the LED die. Such an optical gel is available from Nye Lubricants of Fairhaven, Mass., USA. (It is believed that Nye Lubricants is a name under which the company known, or formerly known, as William F. Nye, Inc. of New Bedford, Mass., or a related company thereof, trades).

In the region of the free end of the tube, the gel is covered by a layer of hardened transparent epoxy resin that provides optical lensing, physical protection and some refractive index matching between the semiconductor dice and the outside atmosphere. An insulating layer 46 is placed between the printed circuit board 43 and the reflector 42.

The method of treatment described above with reference to the first embodiment may also be performed by the apparatus according to this second embodiment. The wavelengths and intensity of light emitted by the above-described apparatus are the same as described with reference to the first described embodiment. However, the apparatus of the second embodiment has the advantage that, if desired, the apparatus is able to be used to produce higher levels of light intensity. This advantage may be achieved by lowering the operating temperature of the LED array still further, thus increasing the LED efficiency and also allowing the device to be driven to currents higher than that that would be possible at the higher operating temperatures of the LED array. In devices of the prior art, the current flowing through an LED array causes a temperature rise in the LEDs. The maximum temperature at which the LED will work properly depends on the packaging and wiring of the LED die. Thus, if the base temperature of the LED heat sink is lowered then more current may be passed through the LED before the maximum allowable LED temperature is reached. Of course, there may be other limitations, such as maximum permissible current, but such limitations can be overcome with changes to the packaging of the LED array.

It will be appreciated that various modifications may be made to the above-described embodiments of the invention without departing from the spirit of the invention. For example, the illuminating device used may be in the form of any illuminating device able to produce controlled doses of radiation at appropriate energy levels and wavelengths, without exceeding certain power levels. For example, the illuminating device may be in the form of a line of a plurality of the illuminating devices described above (a “line treatment”) or could be in the form of the 2-D array of devices as proposed for use in the trials (a “wide area treatment”). There may also be provided a lower fluence device for spot treatment or for line treatment that can be used to “top-up” the higher dosage spot treatment described above. Such a low fluence device would be particularly suitable for home-use.

In the embodiments described above, the wavelength of radiation used is in the range 570-600 nm. However, other embodiments are envisaged that target other peaks in the porphyrin absorption within the skin tissue. Thus, for example light radiation having wavelengths in the violet/near ultra-violet light, blue, green and red wavelength bands could also be used, either individually, or in various combinations. The light could be emitted from a single apparatus (possibly from a single illuminating device) or by separate apparatuses. The control unit would of course control the relative levels of light for the different colours to deliver differing amounts of reactive light at different depths in the skin—thereby tailoring the proposed treatment dependent on the depth of infection by the Propionibacterium acnes bacteria.

The spreader of the second embodiment could also be in the form of a shaped heat pipe and could be formed of diamond coated metal. With reference to the second embodiment, rather than modifying the packages of commercially available LEDs by machining their sides, the LED dice could be mounted on a header specifically designed for the purpose. Also, the gel inside the reflector tube could be replaced by a number of gels with different refractive indices so as to shape the output light beam in some desired form, for example to produce a narrower beam than would otherwise be the case. The water cooling system of the second embodiment could of course use a liquid coolant other than water.

1. A method for improving the appearance of a region of skin by means of conducting the steps of
providing an illuminating device, and
operating the illuminating device to direct light radiation on to the region of skin, wherein
during a period of at least 100 ms, said region receives light energy from the illuminating device of at least 0.01 Jcm⁻²,
the light energy so delivered in any given period of 10 μs is less than 0.5 Jcm⁻², and
the light energy so delivered in any given period of 100 ms is less than 5 Jcm⁻².

2. A method according to claim 1, wherein the method is conducted for the purpose of treating a skin condition and wherein
the illuminating device is operated to direct light radiation on to an area of skin affected by the skin condition, and
the light energy so delivered causes a photochemical reaction within the skin affected by the skin condition, the reaction at least partially disabling or destroying agents contributing to the symptoms of the skin condition.

3. A method according to claim 2, wherein the skin condition is Acne Vulgaris.

4. A method according to claim 2, wherein the agents are bacteria.

5. A method according to claim 2, wherein said photochemical reaction stimulates the production of free-radicals, which then react with, and at least partially disable or destroy, agents causing the skin condition.

6. A method according to claim 1, wherein the illuminating device comprises one or more light emitting semiconductor devices.

7. A method according to claim 6, wherein each said semiconductor device is in the form of a diode.

8. A method according to claim 1, wherein the illuminating device delivers a dose of light radiation to an area of skin, the dose being provided over a period of between 200 ms and 10 seconds and the energy of the light radiation delivered during the dose being greater than 0.1 Jcm⁻² and being equal to T₁×P₁, where T₁=the length in time of the dose and P₁ has the units of optical power density (power per unit area) and satisfies 0.2 Wcm⁻²<P₁<20 Wcm⁻².

9. A method according to claim 1, wherein the illuminating device delivers a dose of light radiation to an area of skin, the dose being provided over a period of between 200 ms and 10 seconds and the energy of the light radiation delivered during the dose being equal to T₂×P₂, where T₂=the length in time of the dose and P₂ has the units of optical power density (power per unit area) and satisfies 0.01 Wcm⁻²<P₂<1 Wcm².

10. A method according to claim 8, wherein said region of skin has a surface area of between 12 and 200 mm².

11. A method according to claim 9, wherein said region of skin has a surface area of between 12 and 200 mm².

12. A method according to claim 1, wherein the illuminating device delivers a dose of light radiation to an area of skin, the dose being provided over a period of between 300 ms and 10 hours, the energy of the light radiation delivered during the dose being greater than 0.1 Jcm⁻² and being equal to T₃×P₃, where T₃=the length in time of the dose and P₃ has the units of optical power density (power per unit area) and satisfies 0.5 mWcm⁻²<P₃<500 mWcm⁻².

13. A method according to claim 12, wherein said region of skin has a surface area of between 0.003 and 0.5 m².

14. A method according to claim 1, wherein the illuminating device provides light radiation including radiation having a wavelength between 570 nm and 600 nm.

15. A method according to claim 1, wherein the illuminating device provides light radiation including radiation having a wavelength between 390 nm and 420 nm.

16. A method according to claim 14, wherein the illuminating device provides light radiation including radiation having a wavelength between 390 nm and 420 nm.

17. A method for treating the skin condition Acne Vulgaris, the method including the steps of
providing one or more light emitting diodes, and
operating said one or more light emitting diodes to direct light radiation on to a region of skin affected by Acne Vulgaris, wherein
during a single dose lasting at least 100 ms, said region receives light energy from said one or more light emitting diodes of at least 0.01 Jcm⁻², and
the light energy causes a photochemical reaction in said region that stimulates the production of free radicals that react with, and at least partially disable or destroy, bacteria that contribute to the symptoms of Acne Vulgaris.

18. A method according to claim 17, wherein
the light energy so delivered in any given period of 10 μs is less than 0.5 Jcm⁻², and
the light energy so delivered in any given period of 100 ms is less than 5 Jcm⁻².

19. A method according to claim 17, wherein the light energy so delivered during a single dose is insufficient to cause any significant photothermal reaction in said region.

20. A method according to claim 17, wherein the alleviation in the symptoms of Acne Vulgaris in said region caused by the light energy so delivered is substantially entirely due to free-radicals caused by said photochemical reaction.

21. An apparatus for the treatment of a skin condition comprising
an illuminating device, and
a control unit for controlling the operation of the illuminating device, wherein
the illuminating device is so arranged and configured that it is able in use to emit light radiation of an energy and wavelength profile sufficient to cause a photochemical reaction within an area of skin affected by a skin condition, which reaction would result in agents causing the skin condition being at least partially disabled or destroyed, and
the control unit and illuminating device are so arranged and configured that the control unit is able in use to cause the illuminating device to direct light radiation on to an area within a distance of no more than 1000 mm from the illuminating device such that:
the light energy received at said area during a period of at least 100 ms is at least 0.01 Jcm⁻²,
the light energy received at said area in any given period of 10 μs is less than 0.5 Jcm⁻², and
the light energy so delivered in any given period of 100 ms is less than 5 Jcm⁻², whereby
the apparatus may be used to treat the skin condition.

22. An apparatus according to claim 21, wherein the control unit and illuminating device are so configured and arranged that the control unit is able in use to cause the illuminating device to deliver a single dose of light radiation to an area of skin, the single dose being provided over a period of between 200 ms and 10 seconds and the energy of the light radiation delivered during the single dose being greater than 0.1 Jcm² and being equal to T₁×P₁, where T₁=the length in time of the single dose and P₁ has the units of optical power density (power per unit area) and satisfies 0.2 Wcm⁻²<P₁<20 Wcm⁻².

23. An apparatus according to claim 21, wherein the control unit and illuminating device are so configured and arranged that the control unit is able in use to cause the illuminating device to deliver a single dose of light radiation to an area of skin, the single dose being provided over a period of between 200 ms and 10 seconds and the energy of the light radiation delivered during the single dose being equal to T₂×P₂, where T₂=the length in time of the single dose and P₂ has the units of optical power density(power per unit area)and satisfies 0.01 Wcm⁻²<P₂<1 Wcm².

24. An apparatus according to claim 22, wherein the apparatus is so configured and arranged that, during a single dose of light radiation, an area of skin of between 12 and 200 mm² is treated.

25. An apparatus according to claim 23, wherein the apparatus is so configured and arranged that, during a single dose of light radiation, an area of skin of between 12 and 200 mm² is treated.

26. An apparatus according to claim 21, wherein the control unit and illuminating device are so configured and arranged that the control unit is able in use to deliver, during a single treatment lasting between 300 ms and 10 hours, light radiation to an area of skin, the energy of the light radiation delivered during the single treatment being greater than 0.1 Jcm⁻² and being equal to T₃×P₃, where T₃=the length in time of the single treatment and P₃ has the units of optical power density (power per unit area) and satisfies 0.5 mWcm⁻²<P₃<500 mWcm⁻².

27. An apparatus according to claim 26, wherein the apparatus is so configured and arranged that, during a single treatment, an area of skin of between 0.003 and 0.5 m² is treated.

28. An apparatus according to claim 21, wherein the illuminating device is arranged to provide light radiation including radiation having a wavelength between 570 nm and 600 nm.

29. An apparatus according to claim 21, wherein the illuminating device is arranged to provide light radiation including radiation having a wavelength between 390 nm and 420 nm.

30. An apparatus according to claim 28, wherein the illuminating device is arranged to provide light radiation including radiation having a wavelength between 390 nm and 420 nm.

31. An apparatus according to claim 21, wherein the illuminating device comprises one or more light emitting semiconductor devices.

32. An apparatus according to claim 31, wherein each said semiconductor device is in the form of a diode.

33. An apparatus for the treatment of Acne Vulgaris comprising
one or more light emitting diodes, and
a microprocessor for controlling the operation of said one or more light emitting diodes, wherein
said microprocessor and said one or more light emitting diodes are so arranged and configured that said one or more light emitting diodes are able in use to deliver a dose of light radiation onto a surface during a period of at least 100 ms, such that the surface receives during said dose light energy of at least 0.01 Jcm⁻², the light so delivered including radiation having a wavelength in the range of 350 nm and 700 nm, whereby
the apparatus may be used to treat a region of skin affected by the condition Acne Vulgaris by causing a photochemical reaction in said region that stimulates the production of free radicals that react with, and at least partially disable or destroy, bacteria that contribute to the symptoms of Acne Vulgaris.

34. An apparatus according to claim 33, wherein apparatus is so arranged that the light energy so delivered in any given period of 10 μs is less than 0.5 Jcm⁻², and
the light energy so delivered in any given period of 100 ms is less than 5 Jcm⁻².
